(19) United States
(12) Patent Application Publication
(10) Pub. No.: US 2023/0233248 A1
(43) Pub. Date: Jul. 27, 2023

(54) HANDPIECE FOR A SURGICAL DEVICE

(71) Applicant: GYRUS MEDICAL LIMITED, Cardiff (GB)

(72) Inventors: James Alan Dickson, Cardiff (GB); Salif Allen Ballo, Cardiff (GB); William Rowlands, Cardiff (GB)

(73) Assignee: GYRUS MEDICAL LIMITED, Cardiff (GB)

(21) Appl. No.: 18/158,805

(22) Filed: Jan. 24, 2023

Related U.S. Application Data
(60) Provisional application No. 63/303,754, filed on Jan. 27, 2022.

(51) Int. Cl. A61B 18/14

(52) U.S. Cl. CPC A61B 18/148; A61B 2018/00083

(57) ABSTRACT

A handpiece for a surgical instrument, having a lumen for the provision of suction or fluids to the surgical instrument, the lumen being formed from at least two concentrically arranged tubes, one of the tubes being formed from metal, and the other of the tubes being formed from an electrically insulating material, the arrangement being such as to prevent electrical charge carried by any fluid within the lumen from leaking to an external surface of the lumen. In one example the at least two concentrically arranged tubes define a tube connector having a distal end and a proximal end, wherein the proximal end is located within a receiving formation in the handpiece and the distal end protrudes outwardly from the handpiece.

### TECHNICAL FIELD

The present disclosure relates to a handpiece for an electrosurgical device.

### BACKGROUND

Electrosurgical instruments, such as may be used in laparoscopic surgical procedures where access to the surgical site is restricted to a narrow passage, often comprise an end effector disposed on the end of an elongate shaft. The end effector may include, for example, elements such as a gripper, scalpel, clamp, components for performing tissue removal, and/or a lumen for providing irrigation and/or suction to a surgical area, so that a single device can be used to perform multiple tasks at the surgical site without having to insert and remove multiple instruments through the narrow passage. The end effector and shaft are typically removably connectable to a handpiece having an electric motor for driving elements of the end effector, a controller for controlling the elements, associated electronic components, and a power supply.

The primary standard governing the safety, essential performance and electromagnetic compatibility of electrical medical equipment of this type in Europe is EN 60601 (equivalent to the international standard IEC 60601). Among other requirements, EN/IEC 60601 specifies that the surfaces of electrical medical devices with which users may come into contact must be electrically insulated to protect users from electrocution. Therefore, in order to satisfy the requirements of EN/IEC 60601, the electronic components of the handpiece must be electrically insulated from the external surface. Devices of this type typically include a lumen extending therethrough for conveying fluids to and from a surgical site. Such fluids include blood plasma, saline and other aqueous solutions capable of being charged. Since the device includes electrical components which are connected to a power source during operation, it is essential that the lumen is electrically insulated from the electrical components. It is also essential that the lumen is electrically insulated and sealed to prevent electrical charge carried by a fluid within the lumen from leaking to an external surface of the device.

A conventional method of electrically insulating handpieces of this type involves constructing the external surface, lumen, and tube connectors entirely out of plastic materials (as shown in FIG. 1). However, a problem associated with this method is that plastics are prone to perishing when exposed to high temperature sterilisation processes, and can crack or snap off if the handpiece is dropped. This can be particularly problematic for tube connectors for connecting suction or irrigation tubes to the lumen, since they are typically integrally formed with the handpiece or irreversibly bonded thereto. As a result, if they break it can be difficult to repair or replace them. This can have the effect of rendering handpieces unusable once their connectors are broken or damaged. There is therefore a need for a handpiece which is capable of overcoming the aforementioned problems, at least to some extent.

### SUMMARY OF THE INVENTION

A handpiece for a surgical instrument is described, the handpiece having a lumen for the provision of suction or fluids to the surgical instrument, the lumen being formed from at least two concentrically arranged tubes, one of the tubes being formed from metal, and the other of the tubes being formed from an electrically insulating material, the arrangement being such as to prevent electrical charge carried by any fluid within the lumen from leaking to an external surface of the lumen. In one example the at least two concentrically arranged tubes define a tube connector having a distal end and a proximal end, wherein the proximal end is located within a receiving formation in the handpiece and the distal end protrudes outwardly from the handpiece.

In accordance with a first aspect of the invention, there is provided a handpiece for a surgical instrument, the handpiece having a lumen for the provision of suction or fluids to the surgical instrument, the lumen being formed from at least two concentrically arranged tubes, one of the tubes being formed from metal, and the other of the tubes being formed from an electrically insulating material, the arrangement being such as to prevent electrical charge carried by any fluid within the lumen from leaking to an external surface of the lumen.

The handpiece provides an electrically insulated surgical device which complies with the requirements of industry standards EN/IEC 60601 and ensures that a user is protected from electrocution. The use of a metal tube advantageously reduces the likelihood of a crack or perforation developing in the lumen through which liquid may leak to the external surface.

The at least two concentrically arranged tubes may define a tube connector having a distal end and a proximal end, wherein the proximal end may be located within a receiving formation in the handpiece and the distal end may protrude outwardly from the handpiece. The distal end provides a connection point for a tube or hose which can be used to provide liquids or suction to the lumen. The receiving formation enables the at least two concentrically arranged tubes to be secured to the handpiece during manufacturing.

An electrically insulating adhesive may be provided between the receiving formation and the lumen. This provides enhanced sealing between the receiving formation and the tubes and ensures that fluid within the lumen is prevented from leaking to the external surface between the opposing surfaces of the tubes and receiving formation.

The tube formed from electrically insulating material may be surrounded by the tube formed from metal. In this arrangement, the metal tube provides structural support to the tube formed from electrically insulating material and protects it from cracking or snapping if dropped or otherwise contacted with force. In this respect, the metal tube is of sufficient strength and thickness to perform these tasks, and is generally of rigid construction that does not bend appreciably under the usual forces encountered in normal use. The thickness of the metal tube material is therefore greater than that of a typical foil material, for example typically greater than 1 mm, and more preferably greater than 1.5 mm or 2 mm, and preferably provides a break force in an example cantilevered stress test of greater than 150 N, and more preferably greater than 200 N

The tube formed from electrically insulating material may extend into the receiving formation to a greater extent than the tube formed from metal. In this arrangement, a barrier is created between the metal tube and lumen and prevents current from passing from electrically charged fluids which may be present in the lumen to the metal tube. Due to the high electrical conductivity of metals it is essential that the metal tube is electrically isolated from either or both of the lumen and external surface of the handpiece, so that current cannot leak to an external surface.

The electrically insulating material may comprise a polymer selected from the group consisting of polyether ether ketone (PEEK), polyethylene (PE), polyvinylchloride (PVC), polypropylene (PP), polyamide (PA), polyester-resin, phenol resin, silicon resin, and epoxy resin, preferably PEEK. These are all examples of electrically insulating polymers. PEEK is particularly preferred because it is tough and resistant to chemical and hydrolytic degradation even at the high temperatures typically employed in medical sterilisation processes.

The polymer may be reinforced with glass fibres. The combination of the polymer and glass fibres produces a composite which has advantageous strength properties and reduced levels of wear, cracking and perishing by comparison with non-reinforced polymers.

The tube formed from metal may be surrounded by the tube formed from electrically insulating material. The tube formed from an electrically insulating material provides a barrier between the metal tube, which may become electrically charged through contact with an electrically charged fluid in the lumen, and an external contactable surface of the handpiece.

The tube formed from metal may extend into the receiving formation to a greater extent than the tube formed from electrically insulating material. By securing the metal tube within the receiving formation, the lumen is strengthened against lateral forces which may otherwise crack or snap the tube or cause the tube to become dislodged from the handpiece. The tube formed from electrically insulating material is provided for electrical insulation rather than strength, and it is not necessary that it extend into the receiving formation to the same extent as the metal tube, or even at all.

The tube formed from electrically insulating material may not extend into the receiving formation. By reducing the quantity of electrically insulating material used, the manufacturing costs of the handpiece may be reduced.

The metal may comprise stainless steel, chrome, nickel, copper or zinc, preferably stainless steel. All of the foregoing metals are capable of providing a reinforcing strength to the lumen.

The stainless steel may be austenitic 316 stainless steel. This metal alloy provides excellent resistance to corrosion and cracking. It is also a non-magnetic form of stainless steel, which makes it ideal for use in electronic medical devices.

An external surface of the handpiece may comprise a polymer selected from the group consisting of polyether ether ketone (PEEK), polyethylene (PE), polyvinylchloride (PVC), polypropylene (PP), polyamide (PA), polyester-resin, phenol resin, silicon resin, and epoxy resin, preferably PEEK. These polymers are non-conductive and provide electrical insulation to the external surface of the handpiece.

The surgical instrument may be a radio frequency (RF) electrosurgical device. The electrosurgical device may be an arthroscopic shaver. These types of surgical instruments employ RF electrical current when operational and include a lumen through which conductive fluids may be conveyed. The features of this aspect of the invention serve to electrically insulate the lumen from the external surface to ensure compliance with industry standards EN/IEC 60601. Advantageously, the features of this aspect also ensure that the lumen is strengthened against wear and tear so that the handpiece has a prolonged lifetime.

In accordance with a second aspect of the invention, there is provided a surgical instrument comprising the handpiece as defined above, a shaft distal to the handpiece, and an end effector at a distal end of the shaft.

The surgical instrument may be an electrosurgical instrument.

In accordance with a third aspect of this invention, there is provided a powered surgical tool system comprising a power source and a surgical instrument as defined above.

The advantages associated with the second and third aspect of the invention correspond to those of the first aspect defined above.

### BRIEF DESCRIPTION OF THE DRAWINGS

Embodiments of the invention will now be further described by way of example only and with reference to the accompanying drawings, wherein like reference numerals refer to like parts, and wherein:

FIG. 1 is a prior art handpiece comprising a hose tail connector made entirely out of plastic;

FIG. 2 is a sectional view of a first embodiment of the invention showing a lumen comprising a metal tube surrounding a tube formed from electrically insulating material;

FIG. 3 is a sectional view showing the lumen of FIG. 2 secured to a handpiece;

FIG. 4 is a sectional view of a second embodiment of the invention showing a lumen formed from a metal tube which is surrounded by a tube formed from electrically insulating material;

FIG. 5 is a sectional view showing the lumen of FIG. 4 secured to a handpiece;

FIG. 6 is a sectional view of the second embodiment showing the outer electrically insulating tube covering a reduced portion of the metal tube;

FIG. 7 is a schematic representation of a surgical instrument comprising the handpiece of the present invention;

FIG. 8 illustrates a perspective view of a powered surgical tool system that incorporates the surgical instrument of FIG. 7, a control unit, a fluid source, a suction source and a power source.

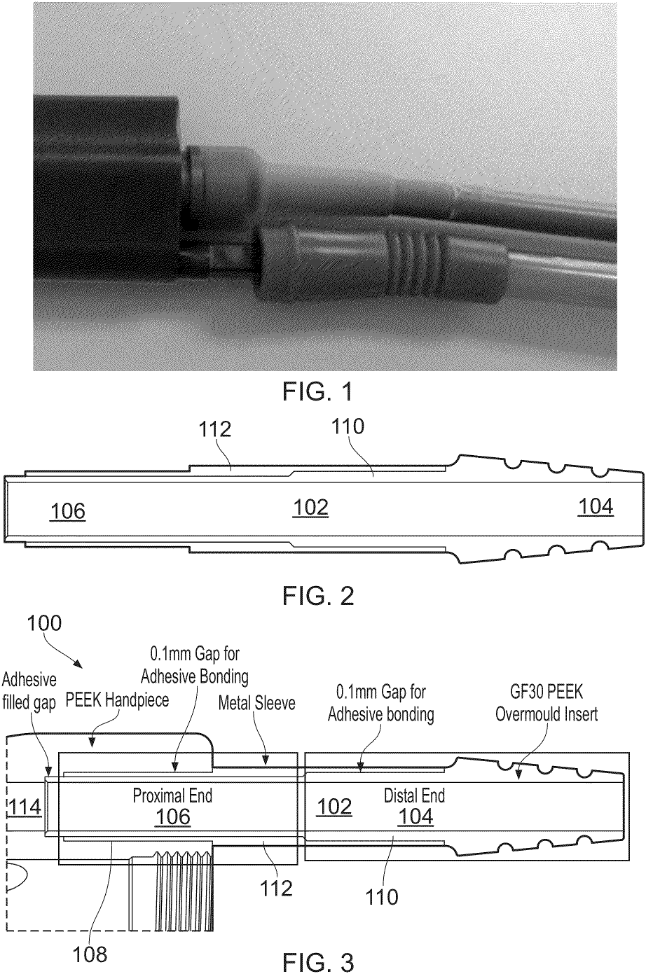

### DETAILED DESCRIPTION

A handpiece for a surgical instrument is provided. The handpiece comprises a lumen for the provision of suction or fluids to the surgical instrument which is formed from at least two concentrically arranged tubes in which one of the tubes is formed from metal and the other of the tubes is formed from an electrically insulating material. The tubes are arranged to prevent electrical charge carried by any fluid within the lumen from leaking to an external surface of the lumen, thereby protecting a user from electrocution. The handpiece typically forms part of an electrosurgical instrument or system, such as may be used to perform laparoscopic (keyhole) surgery. For example, the electrosurgical instrument may be an arthroscopic shaver.

The electrically insulating material and/or an external surface of the handpiece may comprise a polymer selected from the group consisting of polyether ether ketone (PEEK), polyethylene (PE), polyvinylchloride (PVC), polypropylene (PP), polyamide (PA), polyester-resin, phenol resin, silicon resin, and epoxy resin. In a preferred embodiment, the electrically insulating material is PEEK. Preferably, the polymer has a dielectric strength in a range of from 1 to 100 Kv/mm, preferably from 5 to 50 Kv/mm, from about 10 to 30 Kv/mm, or less than 50 Kv/mm, less than 40 Kv/mm, less than 30 Kv/mm, less than 25 Kv/mm, e.g. about 21.5 Kv/mm, for a 2 mm thick layer when tested in a ASTM D149 or IEC 60243-1 standard test. The electrically insulating material is selected to have a dielectric strength, thickness and geometry which is sufficient to reduce RF leakage current to the allowable levels specified in IEC 60601 standards. The polymer may be reinforced with glass fibres to strengthen the electrically insulating material. For example, the polymer and glass fibres may be combined to form a composite material (e.g. GF30 PEEK) which has advantageous strength properties and reduced susceptibility to cracking by comparison with non-reinforced polymers.

The metal from which the metal tube is formed may be selected from the group consisting of stainless steel, chrome, nickel, copper or zinc. In a preferred embodiment, the metal is stainless steel. The stainless steel may be austenitic 316 stainless steel, which provides resistance to corrosion and cracking and is a non-magnetic form of stainless steel. This makes it particularly suited for use in electrical medical equipment.

As shown in FIGS. 2 to 6, the at least two concentrically arranged tubes define a tube connector having a distal end 104 and a proximal end 106. The proximal end 106 is located within and secured to a receiving formation 108 in the handpiece 100 and the distal end 104 protrudes outwardly therefrom. The receiving formation 108 and proximal end 106 are shaped to engage each other in a complementary male and female-type connection. In the embodiments shown in FIGS. 2 to 6, the distal end 104 defines a hose tail connector to which a tube or hose for conveying fluid and suction to and from the lumen may be connected.

As shown in FIGS. 2 and 3, a first embodiment of the handpiece 100 comprises a lumen 102 formed from two concentrically arranged tubes 110, 112 in which an inner tube 110 formed from an electrically insulating material is surrounded by an outer tube 112 formed from a metal. The lumen 102 is defined within the tube 110 formed from electrically insulating material and is in fluid communication with a passage 114 in the handpiece through which fluid or suction may be provided to a surgical area. Both tubes 110, 112 extend into the receiving formation 108 and are secured therein. An electrically insulating adhesive (e.g. a cyanoacrylate/epoxy hybrid) is provided between the receiving formation 108 and the outer tube 112 (and optionally also between the receiving formation 108 and the inner tube 112) to seal the proximal end 106 within the receiving formation 108 and prevent leakage of fluid from the lumen 102 to the external surface. The tube 110 formed from electrically insulating material extends further into the receiving formation 108 than the tube 112 formed from metal to provide a barrier between the lumen 102 and metal tube 112, effectively isolating any current path in the lumen 102 from the metal tube 112 and external surface. The metal tube 112 fits within the receiving formation 108 with a clearance of about 0.1 mm to ensure a strong fit and to provide a gap which can be filled with the adhesive. Adhesive is also provided between the two tubes to prevent ingress of charged fluids therebetween. A clearance of about 0.1 mm may be provided between the tubes to accommodate the adhesive.

The tube 110 formed from electrically insulating material includes a thickened side wall proximate the distal end 104 to enhance its load-bearing capacity and reduce the likelihood of failure in the event of a lateral force being applied to it, such as may occur in use when a tube or hose is connected to the tube connector and the handpiece is moved in a lateral or rotational direction.

The proximal end 106 is provided with a sloped edge (e.g. a chamfer of H7/H6) to provide a precise sliding fit with the receiving formation 108. Adhesive is provided between the receiving formation 108 and sloped edge to prevent leakage of fluids.

The embodiment of the handpiece 100 shown in FIGS. 2 and 3 has been found to have a break force of 200N when evaluated in a cantilever bending stress test. This is considerably higher than the corresponding break force of a handpiece of the prior art having a hose tail connector constructed entirely from plastic of a similar geometry which was found to have a break force of only 95N.

In addition to having a higher break strength, the embodiment shown in FIGS. 2 and 3 has an improved and higher quality feel to users because the outer, visible tube is formed from a metal rather than a plastic. Furthermore, the use of a tube formed from a glass fibre reinforced polymer (e.g. PEEK glass fibre composite) as the electrically insulating material provides enhanced resistance to cracking when evaluated in thermal expansion tests.

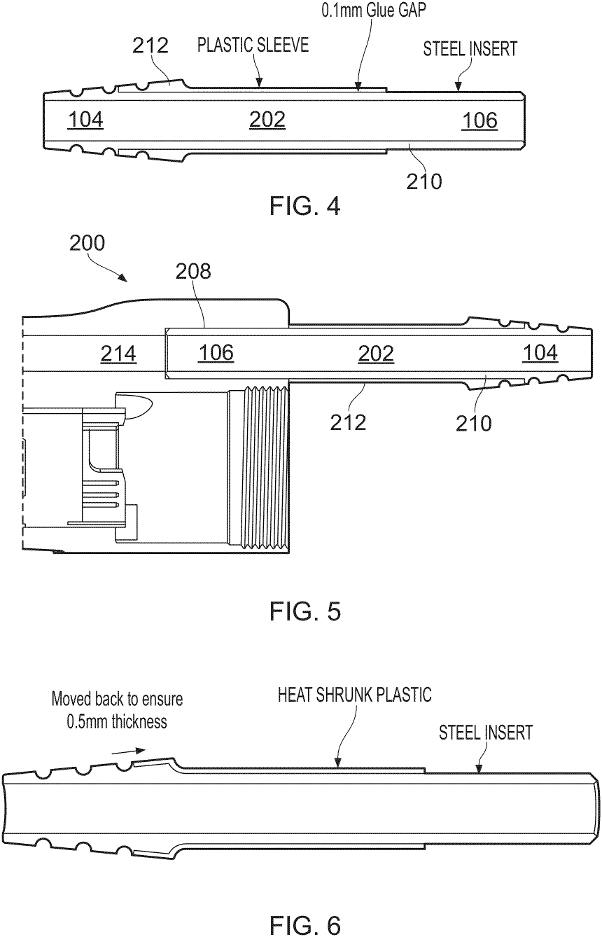

As shown in FIGS. 4 to 6, a second embodiment of the handpiece 200 comprises a lumen 202 formed from two concentrically arranged tubes in which an inner tube 210 formed from a metal is surrounded by an outer tube 212 formed from an electrically insulating material. As shown in FIG. 5, the lumen 202 is defined within the tube 210 formed from a metal and is in fluid communication with a passage 214 in the handpiece 200 through which fluid or suction may be provided to a surgical area. The distal end 104 of the metal tube 210 defines a tube connecting portion, which in the embodiment shown in FIGS. 4 to 6, is a hose tail connector. The metal tube 210 extends into and is secured within a receiving formation 208 in the handpiece 200. Electrically insulating adhesive (e.g. a cyanoacrylate/epoxy hybrid) is provided between the metal tube 210 and receiving formation 208 to prevent leakage of fluids therebetween. In a similar manner to the first embodiment, the metal tube 210 fits within the receiving formation 208 with a clearance of about 0.1 mm to ensure a strong fit and to provide a gap which can be filled with the adhesive.

In the embodiment shown in FIGS. 4 to 6, the tube 212 formed from electrically insulating material is provided by a plastic sleeve which covers the metal tube 210 from the receiving formation 208 to the hose tail connector at the distal end 104 and provides a barrier between the metal tube 210, which may become electrically charged through contact with an electrically charged fluid in the lumen 202, and an external contactable surface of the handpiece 200. In some embodiments, the plastic sleeve may be shrink fitted, over moulded or glued onto the metal tube 210.

Since the hose tail connector is covered by a hose or tube in use, which will typically be constructed from non-electrically conducting material, it is not necessary that the tube 212 formed from electrically insulating material covers the entire metal tube 210. However, in some embodiments, the tube formed from electrically insulating material extends from the receiving formation 208 to at or near the tip of the distal end 104.

By having the entire tube 210 extending from the receiving formation 208 to the hose tail connector formed from metal, the lumen is strengthened by comparison with lumens of the prior art and the likelihood of tube failure in the event of a lateral force being applied is greatly diminished. Advantages of the second embodiment therefore include enhanced strength and rigidity of the connector, a higher quality feel due to the use of a metal tube, at least part of which is visible to a user, and effective prevention of electrical charge carried by fluids within the lumen from leaking to an external surface.

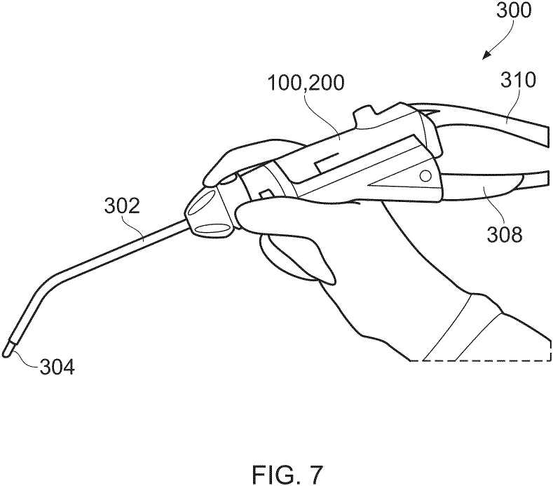

The disclosure extends to a surgical instrument 300 comprising the handpiece 100, 200 as defined above. The surgical instrument 300 may be an electrosurgical instrument. In the embodiment illustrated in FIG. 7, the instrument 300 includes a shaft 302 distal to the handpiece 100, 200, and an end effector 304 at a distal end of the shaft 302. A power cable 308 connected to a power source (not shown), and a tube 310 for providing suction, are also provided.

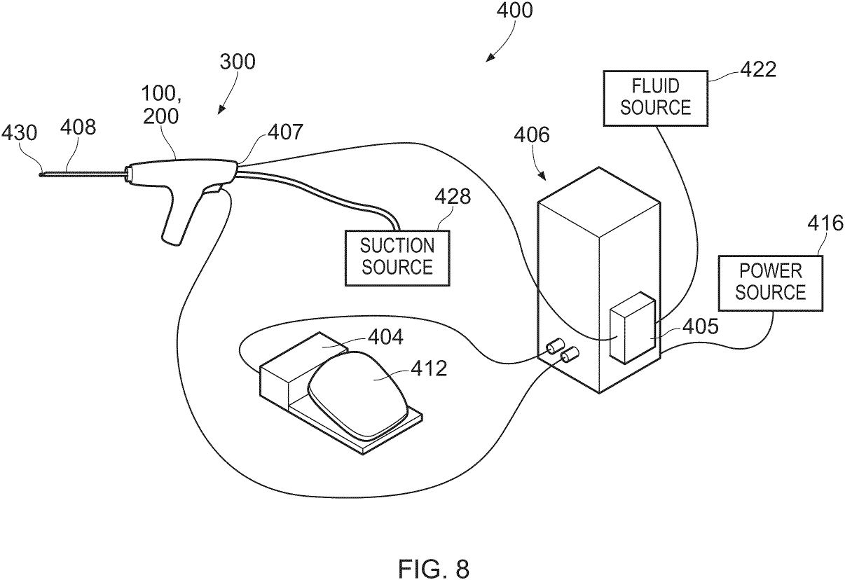

The disclosure extends to powered surgical tool system 400, as shown in FIG. 8, comprising a surgical instrument 300 as defined above. The powered surgical tool system 400 includes a surgical instrument 300 having a handpiece 100, 200, a footswitch 404 (with pedal 412), fluid (liquid and/or gas) source 422, suction source 428, a control unit 406, fluid pump 405 and a fluid inlet/irrigation outlet 407. The system is supplied with power from a power source 416 such as a wall outlet. The suction source 428 may be an external suction source such as provided by attachment to a facility suction outlet provided on a wall. The handpiece 100, 200 is connected, at its distal end, to a shaft 408 including an end effector 430 at its distal end that is used, for example, to cut, shave, remove, resect and/or abrade tissue, bone and/or other bodily materials.

Further modifications, whether by addition, deletion or substitution will be apparent to the intended reader, to provide further embodiments, any and all of which are intended to be encompassed by the appended claims.

1. A handpiece for a surgical instrument, the handpiece having a lumen for the provision of suction or fluids to the surgical instrument, the lumen being formed from at least two concentrically arranged tubes, one of the tubes being formed from metal, and the other of the tubes being formed from an electrically insulating material, the arrangement being such as to prevent electrical charge carried by any fluid within the lumen from leaking to an external surface of the lumen.

2. The handpiece of claim 1, wherein the at least two concentrically arranged tubes define a tube connector having a distal end and a proximal end, wherein the proximal end is located within a receiving formation in the handpiece and the distal end protrudes outwardly from the handpiece.

3. The handpiece of claim 2, wherein an electrically insulating adhesive is provided between the receiving formation and the lumen.

4. The handpiece of claim 1, wherein the tube formed from electrically insulating material is surrounded by the tube formed from metal.

5. The handpiece of claim 2, wherein the tube formed from electrically insulating material extends into the receiving formation to a greater extent than the tube formed from metal.

6. The handpiece of claim 1, wherein the electrically insulating material comprises a polymer selected from the group consisting of polyether ether ketone (PEEK), polyethylene (PE), polyvinylchloride (PVC), polypropylene (PP), polyamide (PA), polyester-resin, phenol resin, silicon resin, and epoxy resin.

7. The handpiece of claim 6, wherein the polymer is reinforced with glass fibres.

8. The handpiece of claim 1, wherein the tube formed from metal is surrounded by the tube formed from electrically insulating material.

9. The handpiece of claim 1, wherein the tube formed from metal extends into the receiving formation to a greater extent than the tube formed from electrically insulating material.

10. The handpiece of claim 2, wherein the tube formed from electrically insulating material does not extend into the receiving formation.

11. The handpiece of claim 1, wherein the metal comprises stainless steel, chrome, nickel, copper or zinc.

12. The handpiece of claim 11, wherein the stainless steel is austenitic 316 stainless steel.

13. The handpiece of claim 1, wherein an external surface of the handpiece comprises a polymer selected from the group consisting of polyether ether ketone (PEEK), polyethylene (PE), polyvinylchloride (PVC), polypropylene (PP), polyamide (PA), polyester-resin, phenol resin, silicon resin, and epoxy resin.

14. The handpiece of claim 1, wherein the surgical instrument is an electrosurgical device.

15. The handpiece of claim 14, wherein the electrosurgical device is an arthroscopic shaver.

16. A surgical instrument comprising a handpiece, a shaft distal to the handpiece, and an end effector at a distal end of the shaft, wherein the handpiece has a lumen for the provision of suction or fluids to the surgical instrument, the lumen being formed from at least two concentrically arranged tubes, one of the tubes being formed from metal, and the other of the tubes being formed from an electrically insulating material, the arrangement being such as to prevent electrical charge carried by any fluid within the lumen from leaking to an external surface of the lumen.

17. The surgical instrument of claim 16, and further comprising an end effector having one or more electrosurgical electrodes thereon for the treatment of tissue with RF energy.

18. An electrosurgical system comprising an RF power source and a surgical instrument, the surgical instrument comprising a handpiece, a shaft distal to the handpiece, and an end effector at a distal end of the shaft, wherein the handpiece has a lumen for the provision of suction or fluids to the surgical instrument, the lumen being formed from at least two concentrically arranged tubes, one of the tubes being formed from metal, and the other of the tubes being formed from an electrically insulating material, the arrangement being such as to prevent electrical charge carried by any fluid within the lumen from leaking to an external surface of the lumen; the surgical instrument further comprising an end effector having one or more electrosurgical electrodes thereon for the treatment of tissue with RF energy.
